(19) United States
(12) Patent Application Publication
(10) Pub. No.: US 2008/0293677 A1
(43) Pub. Date: Nov. 27, 2008

(54) Use of Alternating Amine and Non-Amine Bisphosphonate Combinations For Treating Osteoporosis

(76) Inventor: Kenneth H. Brookler, Norwalk, CT (US)

Correspondence Address: BAKER BOTTS L.L.P., 30 ROCKEFELLER PLAZA, 44TH FLOOR, NEW YORK, NY, 10112-4498, US

(21) Appl. No.: 11/916,619

(22) PCT Filed: Jun. 6, 2005
(86) PCT No.: PCT/US2006/021750

§ 371 Date: Jun. 12, 2008

Related U.S. Application Data
(60) Provisional application No. 60/688,093, filed on Jun. 6, 2005.

(51) Int. Cl. A61K 31/675; A61K 31/663; A61K 31/66; A61P 19/10

(52) U.S. Cl. 514/89; 514/108

(57) ABSTRACT

A method of treating osteoporosis in a human in need thereof by administering alternating amine and non-amine bisphosphonates in a defined dosing schedule. The invention demonstrates an effective response and sustained benefit in the treatment of osteoporosis. Particularly, the method involves administration of a time-dependent dose of more than one bisphosphonate, specifically, alternating administration of a first bisphosphonate with a second bisphosphonate, wherein at least one bisphosphonate is administered in a stepped-up dosage amount.

### CROSS-REFERENCE TO RELATED PRIORITY

This application claims priority from U.S. Provisional Patent Application No. 60/688,093 filed Jun. 6, 2005 which is incorporated by reference herein in its entirety.

### FIELD OF THE INVENTION

The present invention is directed to a method of treating osteoporosis in a human in need thereof with the administration of alternating bisphosphonates, particularly with at least one bisphosphonate in a stepped-up dosage amount. The invention is specifically directed to treating osteoporosis through a dosage regimen that involves alternating amine and non-amine bisphosphonates.

### BACKGROUND OF THE INVENTION

Osteoporosis is a systemic skeletal disease characterized by a low bone mass and microarchitectural deterioration of bone tissue, with a consequent increase in bone fragility and susceptibility to fracture. Because osteoporosis as well as other disorders associated with bone loss are chronic conditions, it is believed that appropriate therapy will generally require chronic treatment.

Osteoporosis affects 20 million people in the United States and leads to an estimated 1.2 million bone fractures annually. It has been estimated that approximately 30 percent of all post-menopausal Caucasian women will suffer from an osteoporotic fracture. Osteoporosis may be treated and/or prevented using a number of different active agents such as antiresorptive agents (e.g., estrogen, selective estrogen receptor modulators, bisphosphonates, and calcitonin) or anabolic agents (e.g., parathyroid hormone). Antiresorptive agents impart skeletal benefits by reducing osteoclastic resorption of bone, thus causing a reduction in bone remodeling and an increase in bone mineral density (BMD). Anabolic agents generally reduce risk of osteoporotic fracture by stimulating new bone formation.

Multinucleated cells called osteoclasts are responsible for causing bone loss through a process known as bone resorption. It is well known that bisphosphonates are selective inhibitors of osteoclastic bone resorption, making these compounds important therapeutic agents in the treatment or prevention of a variety of generalized or localized bone disorders caused by or associated with abnormal bone resorption. Bisphosphonates have been approved by the FDA for use in the treatment of Paget's disease of bone and for osteoporosis.

The compound alendronate is known as a potent bisphosphonate. Evidence suggests that other bisphosphonates such as risedronate, tiludronate, ibandronate, and zolendronate have many properties in common with alendronate, including high potency as inhibitors of osteoclastic bone resorption. Etidronate, an older bisphosphonate, also inhibits bone resorption. However, unlike the more potent bisphosphonates, etidronate impairs mineralization at doses used clinically, and may give rise to osteomalacia, a condition resulting in an undesirable decrease in bone mineralization. See Boyce et al., Lancet 1984, 1(8381):821-824; Gibbs et al., Br. Med. J. 1986, 292:1227-1229.

Despite their therapeutic benefits, bisphosphonates are poorly absorbed from the gastrointestinal tract. When oral administration of bisphosphonates is desired, relatively high doses must be administered to compensate for the low bioavailability from the gastrointestinal tract. See U.S. Pat. No. 5,994,329. To offset this low bioavailability, it is generally recommended that the patient take the bisphosphonate on an empty stomach and fast for at least 30 minutes afterwards. However, fasting on a daily basis can be inconvenient. Moreover, oral administration has been associated with adverse gastrointestinal effects, especially those relating to the esophagus where problems may be exacerbated by the presence of refluxed gastric acid. The degree of adverse gastrointestinal effects of bisphosphonates has been shown to increase with increasing dose. Also, adverse esophageal effects appear to be more prevalent in patients who do not take the bisphosphonate with an adequate amount of liquid or who lie down shortly after dosing, thereby increasing the chance for esophageal reflux.

Current oral bisphosphonate therapies generally fall into two categories: (1) those therapies utilizing continuous daily treatment, and (2) those therapies utilizing a cyclic regimen of treatment and rest periods.

Continuous daily treatment regimens involve the chronic administration of relatively low doses of a bisphosphonate compound with the objective of delivering the desired cumulative therapeutic dose over the course of the treatment period. However, continuous daily dosing has the potential disadvantage of causing adverse gastrointestinal effects due to the repetitive, continuous, and additive irritation to the gastrointestinal tract. Also, because bisphosphonates should be taken on an empty stomach followed by fasting and maintenance of an upright posture for at least 30 minutes, many patients find daily dosing to be burdensome. These factors can therefore interfere with patient compliance, and in some cases require cessation of treatment.

Cyclic treatment regimens were developed because some bisphosphonates, such as etidronate, when given daily for more than several days, have the disadvantage of actually causing a decline in bone mineralization. U.S. Pat. No. 4,761,406 describes a cyclic regimen developed to minimize the decline in bone mineralization while still providing a therapeutic anti-resorptive effect. Generally, cyclic administration is intermittent, as opposed to continuous treatment regimens, and have both treatment periods during which the bisphosphonate is administered and nontreatment periods to permit the systemic level of the bisphosphonate to return to baseline. However, the cyclic regimens, relative to continuous dosing, appear to result in a decreased therapeutic antiresorptive efficacy. Data on risedronate suggests that cyclic dosing is actually less effective than continuous daily dosing for maximizing antiresorptive bone effects. Mortensen et al., J Bone Min. Res., 10(supp. 1): s140 (1995). Furthermore, these cyclic regimens do not eliminate or minimize adverse gastrointestinal effects, because such regimens typically utilize periods of multiple daily dosing.

U.S. Pat. No. 5,366,965 attempts to address the problem of adverse gastrointestinal effects by administering a polyphosphonate compound, either orally, subcutaneously, or intravenously, according to an intermittent dosing schedule having both a bone resorption inhibition period and a no-treatment rest period. However, the regimen has the disadvantage of not being continuous and regular, and requires nontreatment periods ranging from 20 to 120 days.

Based on the current teachings of bisphosphonate regimens, there is a need in the art for a dosing regimen to overcome the shortcomings associated with existing therapies.

### SUMMARY OF THE INVENTION

It has now been discovered in the present invention that alternating administration of more than one bisphosphonate in a defined dosing schedule has a more effective response and sustained benefit in the treatment of osteoporosis.

Accordingly, the invention provides a method of treating osteoporosis in a human in need thereof, comprising alternating administration of two different bisphosphonates, one an amine, the second a non-amine. Preferably, one or the other bisphosphonate is administered in a stepped up dosing schedule, e.g., in a dose that is at least one and a half times the recommended dose for osteoporosis, followed by a second bisphosphonate at the recommended dose. The amine bisphosphonate is administered for a dosage period of at least one week, up to a maximum of 52 weeks, preferably for a dosage period of at least 10 weeks. The non-amine bisphosphonate is administered for a dosage period of at least one week, up to a maximum of 6 weeks. Preferably, the non-amine bisphosphonate is administered for 1-3 weeks. The cycle is repeated for at least about one year, but the invention contemplates administration for at least 5 years, at least 10 years, or until the frequency of bone fracture decreases and bone mineral density is normal.

Administration of the bisphosphonates may be in a single weekly dose, or administration may be more frequent, e.g., administered in divided doses of two, three, four, five, six, or seven times weekly.

In one embodiment, the first bisphosphonate is an amine bisphosphonate and the second bisphosphonate is a non-amine bisphosphonate. In another embodiment, the first bisphosphonate is a non-amine bisphosphonate and the second bisphosphonate is an amine bisphosphonate.

In a specific embodiment, a first bisphosphonate is risedronate and a second bisphosphonate is etidronate. In another alternating dosing schedule, a first bisphosphonate is etidronate and a second bisphosphonate is risedronate.

In yet another embodiment, the method of treating osteoporosis alternates the two different bisphosphonates, wherein the amine bisphosphonate is administered in a stepped up dosing schedule, i.e., in an amount greater than the recommended or approved dose for treating osteoporosis as a single therapeutic. In a specific embodiment, a stepped up dosing schedule is a dose that is at least about one and a half (150%) of the recommended dose for osteoporosis, and the non-amine bisphosphonate is administered in a dosing schedule that is the recommended dose for osteoporosis.

The invention further provides the foregoing methods for administration of alternating bisphosphonates to prevent the development of symptoms of osteoporosis in individuals with a family history of osteoporosis or those determined from other factors to be susceptible to developing the disorder, but not yet diagnosed with symptoms of osteoporosis.

The invention also provides a kit for administering the proper dosage in the alternate dosing schedule treatment. For example, blister packs with alternating dosages can be provided.

The invention also provides for an intravenous dosing schedule of a bisphosphonate for patients unable to tolerate the oral dosing schedules or patients demonstrating no response or intolerance to oral dosing schedules.

These and other aspects of the invention are discussed more in the detailed description and examples.

### DESCRIPTION OF DRAWINGS

FIG. 1 depicts an exemplary timeline of administering alternating bisphosphonates in the present invention.

### DETAILED DESCRIPTION OF THE INVENTION

The present invention advantageously provides a method of treatment of osteoporosis in a human in need thereof. According to the invention, the administration of alternating bisphosphonates in a novel dosage amount significantly lessens adverse symptoms and has a sustained effect. Administration can continue until the frequency of bone fracture diminishes and normal bone density is achieved and maintained.

The terms used in this specification generally have their ordinary meanings in the art, within the context of this invention and in the specific context where each term is used. Certain terms are defined below to provide additional guidance in describing the compositions and methods of the invention.

The term “about” or “approximately” means within an acceptable error range for the particular value as determined by one of ordinary skill in the art, which will depend in part on how the value is measured or determined, i.e., the limitations of the measurement system or on the particular circumstances, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” means within an acceptable error range for the particular value. For example, when referring to a dose of a bisphosphonate, the term “about” refers to the stated amount, plus or minus an acceptable error for an approved product. However, when referring to a period of time, e.g., a week, the present values (20%) are more applicable. Thus, a week can be, e.g., 5 days, 6 days, 8 days, or 9 days, as well as the usual 7 days.

As used herein, the terms “recommended dose for osteoporosis” or “recommended dosing schedule for osteoporosis” refer to any FDA approved dosing schedule, any dosing schedule approved by any other regulatory agency in the United States or abroad, any off-label prescribed dosing schedule for osteoporosis commonly known to those of ordinary skill in the art, or any preferred dosing schedule for osteoporosis appearing in scientific literature.

As used herein, the terms “pharmaceutically acceptable” refer to molecular entities and compositions that are “generally regarded as safe” (GRAS), e.g., that are physiologically tolerable and do not typically produce an allergic or similar untoward reaction, such as gastric upset, dizziness and the like, when administered to an animal. Preferably, as used herein, the term “pharmaceutically acceptable” means approved by a regulatory agency of the Federal or a state government or listed in the U.S. Pharmacopeia or other generally recognized pharmacopeia for use in animals.

### Bisphosphonate

The bisphosphonates of the present invention correspond to the chemical formula,

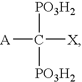

wherein A and X are independently selected from the group consisting of H, OH, halogen, NH₂, SH, phenyl, C₁-C₃₀ alkyl, C₁-C₃₀ substituted alkyl, C₁-C₁₀ alkyl or dialkyl substituted NH₂, C₁-C₁₀ alkoxy, C₁-C₁₀ alkyl or phenyl substituted thio, C₁-C₁₀ alkyl substituted phenyl, pyridyl, furanyl, pyrrolidinyl, imidazonyl, and benzyl.

In the foregoing chemical formula, the alkyl groups can be straight, branched, or cyclic, provided sufficient atoms are selected for the chemical formula. The C₁-C₃₀ substituted alkyl can include a wide variety of substituents, nonlimiting examples which include those selected from the group consisting of phenyl, pyridyl, furanyl, pyrrolidinyl, imidazonyl, NH₂, C₁-C₁₀ alkyl or dialkyl substituted NH₂, OH, SH, and C₁-C₁₀ alkoxy.

In the foregoing chemical formula, A can include X and X can include A such that the two moieties can form part of the same cyclic structure.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

Preferred structures are those in which A is selected from the group consisting of H, OH, and halogen, and X is selected from the group consisting of C₁-C₃₀ alkyl, C₁-C₃₀ substituted alkyl, halogen, and C₁-C₁₀ alkyl or phenyl substituted thio. Preferred structures are those in which A is selected from the group consisting of H, OH, and Cl, and X is selected from the group consisting of C₁-C₃₀ alkyl, C₁-C₃₀ substituted alkyl, Cl, and chlorophenylthio.

Pharmaceutically acceptable salts and derivatives of the bisphosphonates that are also useful herein are included in the definition. Thus, the term etidronate, risedronate, etc., means etidronate or a pharmaceutically acceptable salt thereof, risedronate or a pharmaceutically acceptable salt thereof, etc. Nonlimiting examples of salts include those selected from the group consisting alkali metal, alkaline metal, ammonium, and mono-, di, tri-, or tetra-C₁-C₃₀-alkyl-substituted ammonium. Preferred salts are those selected from the group consisting of sodium, potassium, calcium, magnesium, and ammonium salts. Nonlimiting examples of derivatives include those selected from the group consisting of esters, hydrates, and amides.

It should be noted that the terms “bisphosphonate” and “bisphosphonates”, as used herein, refer to the therapeutic agents of the present invention and are meant to also encompass diphosphonates, biphosphonic acids, and diphosphonic acids, as well as salts and derivatives of these materials, as well as any pharmaceutically acceptable salt of any such agent.

These chemical formulations were initially used for the treatment of the disorder of bone called Paget's disease, for the treatment of hypercalcemia, for the treatment of osteoporosis, and for the prevention of postmenopausal osteoporosis.

Nonlimiting examples of bisphosphonates, include but are not limited to, alendronic acid, 4-amino-1-hydroxybutylidene-1,1-bisphosphonic acid, and Alendronate (also known as alendronate sodium or monosodium trihydrate) (described in U.S. Pat. No. 4,922,007 and U.S. Pat. No. 5,019,651, both of which are incorporated by reference herein in their entirety); Cycloheptylaminomethylene-1,1-bisphosphonic acid, YM 175, Yamanouchi (cimadronate) (as described in U.S. Pat. No. 4,970,335, which is incorporated by reference herein in its entirety); 1,1-dichloromethylene-1,1-diphosphonic acid (clodronic acid), and the disodium salt (clodronate) (described in Belgium Patent 672,205 (1966) and J. Org. Chem 32, 4111 (1967), both of which are incorporated by reference herein in their entirety); 1-hydroxy-3-(1-pyrrolidinyl)-propylidene-1,1-bisphosphonic acid (EB-1053); 1-hydroxyethane-1,1-diphosphonic acid (etidronic acid); 1-hydroxy-3-(N-methyl-N-pentylamino) propylidene-1,1-bisphosphonic acid, also known as BM-210955 (ibandronate) (described in U.S. Pat. No. 4,927,814, which is incorporated by reference herein in its entirety); 6-amino-1-hydroxyhexylidene-1,1-bisphosphonic acid (neridronate); 3-(dimethylamino)-1-hydroxypropylidene-1,1-bisphosphonic acid (olpadronate); 3-amino-1-hydroxypropylidene-1,1-bisphosphonic acid (pamidronate); [2-(2-pyridinyl)ethylidene]-1,1-bisphosphonic acid (piridronate) (described in U.S. Pat. No. 4,761,406, which is incorporated by reference in its entirety); 1-hydroxy-2-(3-pyridinyl)-ethylidene-1,1-bisphosphonic acid (risedronate); (4-chlorophenyl)thiomethane-1,1-disphosphonic acid (tiludronate) (described in U.S. Pat. No. 4,876,248, which is incorporated by reference herein in its entirety); 1-hydroxy-2-(1H-imidazol-1-yl)ethylidene-1,1-bisphosphonic acid (zolendronate); and cimadronate.

Preferred are bisphosphonates selected from the group consisting of alendronate, etidronate, ibandronate, risedronate, pamidronate, zolendronate. As noted above, this includes pharmaceutically acceptable salts thereof. The invention also contemplates mixtures thereof.

Chemicals in this classification with an added amine have the ability to affect osteoclasts by reducing the rate of stimulation of osteoclasts. All chemicals in this classification have the ability to produce programmed cell death of osteoclasts.

In certain embodiments of the invention, the first bisphosphonate is an amine bisphosphonate, for example, alendronate, pamidronate, or residronate. In another embodiment of the invention, the first bisphosphonate is a nonamine bisphosphonate, for example, clodronate or etidronate.

Several bisphosphonates and their recommended dosing schedules for osteoporosis appear in Table 1.

TABLE 1
Recommended Dosing Schedules for Osteoporosis

| | |
|---|---|
| Alendronate | FDA approved dose: Postmenopausal osteoporosis prevention: |
| (Fosamax ®): | 5 mg orally once daily or treatment: 10 mg orally once daily. |
| | Glucocorticoid induced: 5-10 mg orally once daily. |
| | Alendronate should be taken in the morning with 6 to 8 ounces |
| | of water at least one-half hour before food, beverages, or other |
| | medications. |
| Etidronate (Didronel ®): | Recommended by physicians and scientific literature: 400 mg |
| | per day × 2 weeks followed by a period of administration of a |
| | calcium supplement. Adami, S, et al., “Prevention of early |
| | postmenopausal bone loss with cyclical etidronate,” Journal of |
| | Endocrinological Investigation 2000; 23(5): 310-316. |
| Pamidronate (Aredia ®): | Recommended by physicians and scientific literature: 30-90 |
| | mg IV single dose. Wait at least 7 days before considering |
| | retreatment. See Little, DG., “Intravenous pamidronate |
| | reduces osteoporosis and improves formation of the regenerate |
| | during distraction osteobenesis,” Journal Bone & Joint Surgery |
| | 2001, Sept. 83(7): 1069-74, and FDA approved dose for |
| | hypercalcemia. |
| Risedronate (Actonel ®): | FDA approved dose: Osteoporosis prevention and treatment |
| | of: 5 mg orally once daily, 35 mg orally once weekly, or 30 |
| | mg orally once weekly. |
| | Should be taken on an empty stomach in an upright position |
| | with at least 6 ounces of plain water. The upright position and |
| | empty stomach should be maintained for at least 30 minutes to |
| | minimize gastrointestinal adverse events and increase |
| | absorption. |
| Tiludronate (Skelid ®): | Recommended by physicians and scientific literature: 400 mg |
| | orally once daily × 3 months. Each dose should be taken with |
| | 6 to 8 ounces of water. Tiludronate should not be taken within |
| | 2 hours of food or other medications. See Ohnishi H., et al., |
| | “Bisphosphonate tiludronate increases bone strength by |
| | improving mass and structure in established osteopenia ager |
| | ovariectomy in rats,” Bone 1997 Oct. 21(4): 335-43 and FDA |
| | aproved dose for Paget's disease. |
| Ibandronate (Boniva ®) | FDA approved dose: Osteoporosis prevention and treatment |
| | of: 2.5 mg orally once daily. A phase III study has |
| | investigated an intravenous injection in the amount of: 2 mg |
| | every two months and 3 mg every three months. Recently |
| | approved dose is 150 mg per month. |
| Zoledronic acid | Recommended dose: 4 mg zoledronic acid on an anhydrous |
| (Zometa ®) | basis. Note: FDA has not yet approved zoledronic acid for the |
| | treatment of osteoporosis. |

### Alternate Dosing of Bisphosphonates

The present invention involves a method of treating osteoporosis in a human in need thereof, wherein the method comprises alternating administration of different bisphosphonates.

In the present invention, the method of treating osteoporosis involves administering a first bisphosphonate in a stepped up dosage, followed by a second bisphosphonate at a recommended dose, resulting in an unexpected improvement in absorption. Preferably, the stepped up dosage is in an amount greater than the recommended dose for osteoporosis. In specific embodiments, the dosage of the first bisphosphonate administered comprises at least about 150% to about 300% of the recommended dose for osteoporosis. The stepped-up dosing of bisphosphonates of the present invention is calculated herein as at least about 150% of the highest recommended dose for osteoporosis. In the preferred embodiment, the amine bisphosphonate has the stepped up dosage amount. Exemplary stepped up dosing schedules are listed in Table 2.

TABLE 2
Stepped up dosing schedule

| | |
|---|---|
| Alendrolate | 15 mg to 30 mg orally once daily. |
| Pamidronate | 135 mg to 270 mg IV single dose. |
| Risedronate | 45 mg to 90 mg orally per week. |
| Ibandronate | 3.75 mg to 7.5 mg orally once daily. |

In one specific embodiment, the first bisphosphonate is the amine bisphosphonate, risedronate, and the second bisphosphonate is a nonamine bisphosphonate, etidronate or tiludronate.

In an alternate dosing schedule, for example, (1) the first bisphosphonate may be administered in a stepped-up dose of the recommended dose for osteoporosis, e.g., about 150% to 300% of the recommended dose for osteoporosis, while the second bisphosphonate is administered in accordance with the recommended dosing schedule for osteoporosis; or (2) the first bisphosphonate may be administered in accordance with the recommended dosing schedule for osteoporosis while the second bisphosphonate is administered in a dose that is at least 150% of the recommended dose for osteoporosis or 150% to 300% of the recommended dose for osteoporosis.

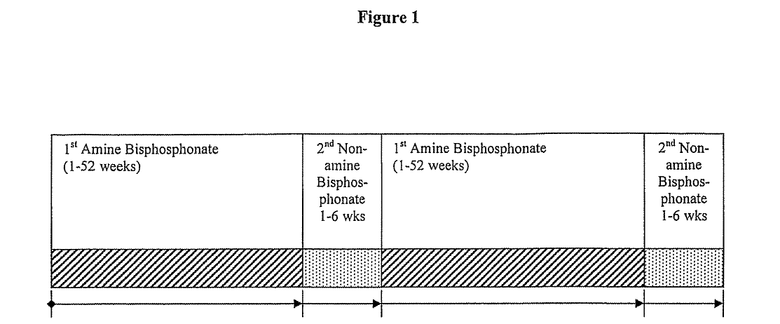

In the present invention, the first bisphosphonate is administered for a first dosing period. In a preferred embodiment, the first dosing period is at a minimum of about 1 week, up to a maximum of about 52 weeks. Preferably, the first bisphosphonate is administered for a first dosing period of a minimum of about 10 weeks to a maximum of about 20 weeks. Once administration of the first bisphosphonate is discontinued, the second bisphosphonate is then administered for a second dosing period. In a preferred embodiment, the second dosing period is at a minimum of about 1 week, up to a maximum of about 6 weeks, preferably from about 1 to about 3 weeks. In a preferred embodiment, the first bisphosphonate is administered for about 13 weeks, followed by administration of a second bisphosphonate for about 3 weeks. The cycle is then repeated until the target endpoints are achieved. See FIG. 1.

In one embodiment of the invention, the alternating dosing schedule continues for at least one year, preferably at least 5 years, more preferably for at least 10 years. Since detection of osteoporosis is measured by endpoints such as frequency of bone fracture and/or normal bone mineral density, which can only be reviewed annually, the dosing regimen continues until there is a decrease in fractures and/or normal bone density. In the event that these endpoints are achieved, treatment may be continued as a prophylactic measure. In one embodiment of the invention, the doses may be stepped down, either by concentration, or with increasing the time period between administrations of the second alternating bisphosphonate. If treatment is stopped, and the patient shows signs of recurrence, the alternating dosage regimen is reinstituted.

In a specific embodiment of the alternate dosing schedule treatment, the administration of one bisphosphonate, risedronate, comprises a stepped up dosage total of at least about 45 mg to about 105 mg per week, preferably at least about 50 mg to 70 mg per week, for at least 1 to 52 weeks, preferably 2 to 26 weeks, followed by administration of the second bisphosphonate, such as etidronate, comprising a total of at least about 400 mg per week, preferably from about 600 mg to about 2800 mg per week, for 1 to 3 weeks, every 6 to 13 weeks.

In another embodiment, the administration risedronate comprises a total of at least about 5 mg to 35 mg, preferably at least about 5 mg to 30 mg per week, for at least 1 to 52 weeks, preferably 2 to 26 weeks, followed by administration of etidronate comprising a total of at least about 600 mg to 2800 mg per week, for 1 to 3 weeks, every 6-13 weeks.

Administration of each of the bisphosphonates may be in a single weekly dose, or the total weekly dosage may be administered periodically in divided doses of two, three, four, five, six, or seven times weekly. Preferably, the bisphosphonates are administered at least one hour before eating and drinking (other than water) after an appropriate fasting period. The bisphosphonates are also preferably administered in a dosage amount and scheduled time so as to achieve optimal absorption and achieve an optimal effect.

In a further embodiment, the alternate dosing schedule also includes continuous supplementation of calcium and Vitamin D and/or calcium and fluoride.

In the present invention, administration of bisphosphonates may be oral or intravenous. Preferably, the administration is oral.

Nevertheless, the present invention provides for intravenous administration of a bisphosphonate alternating with an orally administered bisphosphonates. Intravenous bisphosphonates include, for example, but are not limited to ibandronate, pamidronate, and zolendronate. An intravenous dosing schedule will depend on the recommended dose of the bisphosphonate to be administered by intravenous route. For example, an intravenous dosing schedule for zolendronic acid may be from about 2 mg to about 6 mg, preferably from about 3 mg to about 5 mg, administered from about 2 to about 10 times a year. Preferably, intravenous administration is in periodic dosage amounts as needed so as to achieve an optimal effect. Preferably, the supplemental treatment, e.g., with one or more of calcium, fluoride, Vitamin D, or combinations thereof; or an orally administered bisphosphonate is administered as needed. The supplemental orally administered bisphosphonate may be etidronate, risedronate, alendronate, or tiludronate.

### Kits

The present invention conveniently provides kits for administration of the alternating dosing schedule of two bisphosphonates as set forth above. For example, the kit can comprise a 1 to 52 week, preferably a 2 to 26 week, dose of one bisphosphonate, alternating with a 1 to 6 week dose, preferably a 2 to 4 week dose, of the second bisphosphonate. Instructions for administering the alternating dose may be included in the kit, or as part of the packaging of the kit.

Numerous kit formats are available. For example, a bubble pack, with unit doses of the bisphosphonates, can be provided. The bisphosphonates can be in pill, tablet, capsule, or other solid dosage form, with alternating or the same colors. Each dosage period can be arranged in rows or columns, or the doses can be arranged in a circle to be administered on the appropriate basis. In a circular pattern it may not be necessary to visually distinguish the two alternative bisphosphonates.

To ensure compliance with dosing, it may be advisable to provide a daily dose. In this case, if the optimal dosing regiment is less frequently than daily, identical-looking placebos can be included in the kit to be administered on the non-dosing days to maintain the regular doing pattern, and thus compliance.

Other kits, which may be appropriate for patients on many medications who arrange their medications on a weekly or other periodic schedule, may include two separate vials of oral dosage forms for the subject to arrange for the following week. Preferably such a kit also contains instructions.

### EXAMPLES

The present invention will be better understood by reference to the following Examples, which are provided as exemplary of the invention, and not by way of limitation.

### Example 1

### Method of Treating Osteoporosis

The present example demonstrates a detailed dosing scheme for the treatment of osteoporosis with the use of alternating bisphosphonates.

Patients are screened for osteoporosis and medial history is obtained, specifically regarding frequency of bone fractures and bone mineral density. Each patient is given risedronate in a stepped up dosage for 11 weeks. The dose for the risedronate is about 50 mg to 70 mg per week. Beginning at the 12^{th} week, the administration of risedronate stops and each patient is given a fixed dose of 400 mg etidronate for 2 weeks.

At the end of the 2-week period, each patient starts another 11 week course of the risedronate. Again, at the end of the 11^{th} week, the patient is switched to etidronate for 2 weeks. The cycle is continued for five years.

At the end of each year, medical history is obtained regarding the frequency of bone fracture and the bone mineral density. At the 5 year testing period, if the number of fractures has not decreased and the bone mineral density is not normal, the alternating bisphosphonate treatment is continued for 5 additional years, at which time the same assessment will be carried out. If, however, there is cessation of bone fracture and normal bone density, the use of etidronate may decrease to 1 week, and the course of risedronate will stay the same. Alternatively, the risedronate course will be longer, about 13 to 26 weeks, before alternating to etidronate for 2 weeks. Another alternative is to maintain the dosing schedule, but to decrease the risedronate to 30 to 35 mg once a week. Whichever modified course is selected, the revised treatment is continued for one year. If, at the end of one year, the end points are met, the treatment will either be stopped or the treatment is continued indefinitely as a preventative measure.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, procedures, and the like are cited throughout this application, the disclosures of which are incorporated herein by reference in their entireties.

What is claimed:

1. A method of treating osteoporosis in a human in need thereof, which method comprises alternating administration of bisphosphonate compounds, wherein a first bisphosphonate compound is administered for a first dosing period, followed by a second bisphosphonate administered for a second dosing period.

2. The method of claim 1, wherein the first bisphosphonate is an amine bisphosphonate.

3. The method of claim 1, wherein the first bisphosphonate is a non-amine bisphosphonate.

4. The method of claim 2, wherein the second bisphosphonate is a non-amine bisphosphonate.

5. The method of claim 3, wherein the second bisphosphonate is an amine bisphosphonate.

6. The method of claim 1, wherein the first dosing period ranges from a minimum of about 1 week, to a maximum of about 52 weeks.

7. The method of claim 6, wherein the first bisphosphonate is administered from about 10 to about 20 weeks.

8. The method of claim 6, wherein the first bisphosphonate is administered for about 13 weeks.

9. The method of claim 1, wherein the second dosing period ranges from a minimum of about 1 week, to a maximum of about 6 weeks.

10. The method of claim 9, wherein the second bisphosphonate is administered from about 1 to about 3 weeks.

11. The method of claim 1, wherein the first bisphosphonate is an amine bisphosphonate selected from the group consisting of risedronate, pamidronate, alendronate, pharmaceutically acceptable salts thereof, and mixtures thereof.

12. The method of claim 1, wherein the second bisphosphonate is a non-amine bisphosphonate selected from the group consisting of clodronate, etidronate, pharmaceutically acceptable salts thereof, and mixtures thereof.

13. The method of claim 1, wherein the second bisphosphonate is an amine bisphosphonate selected from the group consisting of risedronate, pamidronate, alendronate, pharmaceutically acceptable salts thereof, and mixtures thereof.

14. The method of claim 1, wherein the first bisphosphonate is a non-amine bisphosphonate selected from the group consisting of clodronate, etidronate, pharmaceutically acceptable salts thereof, and mixtures thereof.

15. The method of claim 1, wherein the first bisphosphonate is risedronate, and the second bisphosphonate is etidronate.

16. The method of claim 1, wherein at least one of the bisphosphonates is administered in a stepped up dosage amount.

17. The method of claim 16, wherein the stepped up dosage amount is from about 150% to about 300% of the recommended dose.

18. The method of claim 1, wherein the dosage of the first bisphosphonate is administered at least about 150% of the recommended dose for osteoporosis, and the dosage of the second bisphosphonate is administered at the recommended dose for osteoporosis.

19. The method of claim 1, wherein the dosage of the second bisphosphonate is at least about 150% of the recommended dose for osteoporosis and the dosage of the first bisphosphonate is a recommended dose for osteoporosis.

20. The method of claim 1, wherein the administration further comprises daily dosing of calcium, vitamin D, fluoride, or a combination thereof.

21. The method of claim 1, wherein at least one bisphosphonate is administered orally.

22. The method of claim 21, wherein the second bisphosphonate is administered intravenously.

23. The method of claim 1, wherein the alternating administration continues for at least one year.

24. The method of claim 1, wherein the alternating administration continues for at least five years.

25. The method of claim 1, wherein the alternating administration continues for at least ten years.

26. The method of claim 1, wherein the first bisphosphonate is risedronate.

27. The method of claim 26, wherein the risedronate is administered at a stepped up dosage total of from about 45 mg to about 90 mg per week.

28. The method of claim 26, wherein the risedronate is administered at a stepped up dosage total of from about 50 mg to about 70 mg per week.

29. The method of claim 26, wherein the risedronate is administered for a first dosing period ranging from about 1 week to about 52 weeks.

30. The method of claim 26, wherein the risderonate is administered for a first dosing period from about 10 to about 20 weeks.

31. The method of claim 26, wherein the risderonate is administered for a first dosing period for about 13 weeks

32. The method of claim 1, wherein the second bisphosphonate is etidronate.

33. The method of claim 32, wherein the etidronate is administered at a dosage total of at least 400 mg per week.

34. The method of claim 32, wherein the etidronate is administered at a dosage total of from about 600 mg to about 2800 mg per week.

35. The method of claim 32, wherein the etidronate is administered for a second dosing period ranging from about 1 week to about 6 weeks.

36. The method of claim 32, wherein the etidronate is administered for a second dosing period ranging from about 1 to about 3 weeks.

37. A kit for the administration of a first bisphosphonate and a second bisphosphonate which comprises:
a) a first dosage of the first bisphosphonate; and
b) a second dosage of the second bisphosphonate.

38. The kit of claim 37, wherein the dosage amounts are arranged in a blister-pack in their time-dependant order.

39. The kit of claim 37, wherein the first bisphosphonate is selected from the group consisting of risedronate and a pharmaceutically acceptable salts thereof, and the second bisphosphonate is selected from the group consisting of etidronate, a pharmaceutically acceptable salt of etidronate, tiludronate, and a pharmaceutically acceptable salt of tilundronate.
